(19) United States
(12) Patent Application Publication
(10) Pub. No.: US 2001/0041792 A1
(43) Pub. Date: Nov. 15, 2001

(54) Extraction of growth factors from tissue

(76) Inventors: Russell S. Donda, Alachua, FL (US); John F. Wironen, Alachua, FL (US)

Correspondence Address: Timothy H. Van Dyke, Bencen & Van Dyke, P.A., 1630 Hillcrest Street, Orlando, FL, 32803, US

(21) Appl. No.: 09/776,619

(22) Filed: Feb. 2, 2001

Related U.S. Application Data
(60) Provisional application No. 60/180,067, filed on Feb. 3, 2000.
(60) Provisional application No. 60/200,842, filed on May 1, 2000.
(60) Provisional application No. 60/215,912, filed on Jul. 3, 2000.

(51) Int. Cl. A61K 38/18; C07K 14/615

(52) U.S. Cl. 530/399; 530/397; 514/2

(57) ABSTRACT

Disclosed herein are novel methods of obtaining osteogenic and other growth factor compositions from alternative nonbone sources such as tissue or bone marrow, and methods of using the same. Also disclosed are implants infused with growth factors obtained from the methods disclosed herein.

### BACKGROUND OF THE INVENTION

Growth factors for inducing production of bone (osteogenic growth factors) have been used for a number of years to aid in the treatment of bone defect and injuries, especially in coordination with the implantation of graft material. Osteogenic growth factors have traditionally been recovered from animal or human bone tissue, or produced through recombinant technology. However, the concentration of growth factors in bone is relatively low, quantity of raw tissue material is limited, and the processing methods are very expensive. Accordingly, there is a need to develop alternative means to obtain growth factors that overcome the drawbacks to the current production methods.

### SUMMARY OF THE INVENTION

The subject invention pertains to a novel method of obtaining growth factors that involves extraction of such growth factors from tissue, including, but not limited to, cadaveric tissue. Specifically exemplified herein is a method of extracting osteogenic or other growth factors from human and/or nonhuman blood, bone marrow and/or muscle tissue. Preferably, these growth factors are added to implants comprised of allograft or xenograft tissue, synthetic compositions, or combinations thereof, to increase osteoinductivity of the implant, or used to induce growth of connective tissue using allograft, xenograft, synthetic compositions, or any combination thereof as a carrier for the growth factors. Extraction of growth factors from such tissues provides increased source tissue and will lessen the expense related to recombinant growth factors. The subject methods are less expensive and more efficient than the current techniques used for extraction. Further, bone paste, bone dowels, and all other bone products could be improved by the implementation of the subject growth factors.

### DETAILED DISCLOSURE OF THE INVENTION

The term “tissue” as used herein refers to any animal tissue types including, but not limited to, bone, bone marrow, neural tissue, fibrous connective tissue, cartilage, muscle, vasculature, skin, adipose tissue, blood and glandular tissue or other nonbone tissue. Preferably, tissue used for extraction in accord with the teachings herein, preferably comprises allograft tissue, and more preferably, cadaveric tissue.

The term “animal” as used herein refers to any animal having a vertebrate structure, preferably a mammal, and most preferably a human.

The term “growth factor” as used herein refers to a polynucleotide molecule, polypeptide molecule, or other related chemical agent that is capable of effectuating differentiation of cells. Examples of growth factors as contemplated for use in accord with the teachings herein include a epidermal growth factor (EGF), transforming growth factor-alpha (TGF-alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), and/or platelet derived growth factor (PDGF).

The terms “osteogenic growth factor” or “OGF” are used herein in their broad sense and refer to a polypeptide molecule or other related chemical agent that effectuates the induction of new bone and/or cartilage formation.

In an alternative embodiment, the growth factors obtained by the subject methods, or other means, are infused into a graft tissue, synthetic compositions, or combinations thereof, that are suitable for implantation into a patient in need thereof. The terms “infuse” or “infused” are used herein in their broad sense and are intended to mean any association, infusion, coating or treatment of the implant whereby a substance is allowed to effectuate its intended beneficial effect, whether it be released or whether contact with the implant is maintained. The choice of the implant material will vary depending on the specific application in which the implant is used. Physical and chemical characteristics such as, e.g., biocompatibility, biodegradability, strength, rigidity, interface properties, and even cosmetic appearance may be considered in choosing an implant material. Examples of materials that are used in accord with the teachings herein include, but are not limited to, bone (cortical and/or cancellous), mineralized collagen (see U.S. Pat. No. 5,231,169), Bio Oss, Norian SRS, collagraft, osteoset, hydroxyapatite, bioglass, aluminates, tricalciumphosplate, calcium sulphate and calcium phosplate, polymeric materials such as acrylic ester polymers and lactic acid polymers (see U.S. Pat. Nos. 4,521,909, and 4,563,489), and glycosaminoglycan (GAG) (U.S. Pat. No. 4,505,266). Preferred materials for making the implants are bioceramics, such as calcium phosphate compositions as taught in U.S. Pat. Nos. 5,676,976; 5,650,176; and 6,027,742, the teachings of which are incorporated by reference.

In addition to growth factors, the implants can also be infused with medically/surgically useful substances. In preferred embodiments, the medically/surgically useful substances include, but are not limited to, commercially available bone pastes such as those disclosed in WO98/40113, collagen and insoluble collagen derivatives; gelatin; hydroxyapatite, etc., and soluble solids and/or liquids dissolved therein, e.g., antiviricides, particularly those effective against viruses such as HIV and hepatitis; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin, etc.; amino acids, magainins, peptides, vitamins, inorganic elements, co-factors for protein synthesis; hormones; endocrine tissue or tissue fragments; enzymes such as collagenase, peptidases, oxidases, etc.; polymer cell scaffolds with parenchymal or other cells; surface cell antigen eliminators; angiogenic or angiostatic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, growth factors, growth hormones such as somatotropin; bone digesters; antitumor agents; fibronectin; cellular attractants and attachment agents; immuno-suppressants; permeation enhancers, e.g., fatty acid esters such as laureate, myristate and stearate mono esters of polyethylene glycol, enamine derivatives, alpha-keto aldehydes, etc.; nucleic acids; bioerodable polymers such as those disclosed in U.S. Pat. Nos. 4,764,364 and 4,765,973, and combinations of any of the foregoing. The amounts of such medically useful substances can vary widely with optimum levels being readily determined in a specific case by routine experimentation. Those skilled in the art will readily appreciate appropriate substances to infuse into appropriate implants based on the intended medical application.

The growth factors obtained by the methods herein can be combined with a number of suitably carriers. Such carriers include, but are not limited to, gelatin, glycerol, collagen, amylopectin, agarose, dextran, inulin, hyaluronic acid, cellulose, albumin, cellulose derivitaves such as carboxynethyl cellulose (CMC), other polyhydroxy compounds, biodegradable polymers such as polylactic or polyglycolic acids, polyvinyl compounds, polycoprolactone, other degradable polyesters, polysulfones, polycarbonates, polyolefins, polyphosphasines polyacrylates, polyamides, polycyanoacrylates, and other degradable polymers or a combination thereof.

In an alternative embodiment, graft tissues are treated with Platelet Rich Plasma (PRP), or growth factors isolated from PRP. PRP obtained from autograft blood has been shown to increase the rate of healing of autogenic grafts. Current methods of applying PRP to such grafts involves the removal of blood from a patient (plasmapheresis), centrifuging the blood, drawing off the PRP layer, and applying the PRP to the graft, which all must occur just prior to surgery. There is a need in the art to alleviate the costs and inefficiencies involved with the current methods. Accordingly, in a further embodiment of the subject invention, provided is a method of obtaining an allograft and/or xenograft source of PRP for use in graft implantation. In a specific embodiment, the PRP is obtained by procuring blood from a cadaveric donor (such as by conventional exsanguination techniques) or procuring blood (preferably expired blood as to avoid depletion of blood earmarked for other purposes) from blood banks, and centrifuging the obtained blood to separate the PRP from other blood components via conventional methods. Preferably, PRP is obtained from a cadaveric donor. The isolation of PRP from sources other than autogenous (recipient) allows for the manipulation and use of the PRP well prior to surgery, whereby the inefficient removal and treatment of blood from the recipient is alleviated.

Furthermore, it is generally believed in the art that the beneficial effects of PRP are due to the presence of various growth factors, such as platelet derived growth factor (PDGF), platelet derived angiogenic growth factor (PDAF), platelet derived epidermal growth factor (PDEGF), and transforming growth factor (TGF-beta). Allogenic and/or xenogenic blood provides a vast and untapped source for PRP and growth factors. In a specific embodiment, platelets are isolated from allogenic and/or xenogenic sources as described above, and growth factors are partially purified or purified from these isolated platelets via conventional methods (see, e.g., U.S. Pat Nos. 4,479,896; 4, 861,757; or 4,975,526). As used herein, the term “partially purified” refers to a state of purification above that which is found in nature, or said differently, that is not achievable unless through manipulation by the hand of man. The term “purified” as used herein refers to a state of purification such that in a given sample comprising a given growth factor, the growth factor is 95% or greater, by weight, of the sample. Once they are partially purified or purified, the growth factors can be stored and/or distributed in a lyophilized or frozen form. Accordingly, the subject methods allow for the mass production of implants (autogenic, allogenic, and/or xenogenic) that have been treated with PRP, and/or growth factors isolated therefrom, that are readily usable in implantation surgeries, which also decreases the costs and inconvenience associated with conventional methods.

In a preferred embodiment, growth factors obtained from blood, or any other growth factors obtained from other tissues as previously described above, are placed in an easy to use container such as a bottle, vial, bag, etc. made from glass or plastics, or other suitable materials. Providing the subject growth factors in containers will facilitate the use of the growth factors, for example, for the infusion or other treatment of implants to be implanted into a patient, or for the direct administration of the growth factors into a patient.

### EXAMPLES

### Example 1:

### Extraction of Growth Factors and Preparation for Implantation

A Guanidine extract solution was prepared by dissolving 4 M guanidine hydrochloride (GuHCl) in 50 mM Tris HCl containing 10 mM EDTA, 100 nM beta-Aminohexanoic Acid, 5 mM benzamidine HCl, and 1 mM phenylmethylsulfonyl fluoride in 1 liter of water. The solution was then filtered in 0.2 micron filter.

50 grams of muscle tissue was added to 500 ml of the Guanidine extract solution and blended in blender to form a homogenate mixture. The homogenate mixture was centrifuged for 30 minutes to eliminate particulate matter, thereby leaving a crude extract. The crude extract was transferred to a 5 kD dialysis tube and dialyzed against distilled water with a minimum of 6, 100-fold changes of water (dialysis was performed at 4° C.). After dialysis, the crude extract was lyophilized. The above procedure was also followed to produce extract from bone marrow except that 60 grams of tissue was added to Guanidine extract solution.

A 0.01 N HCl suspension of each extract was made containing approximately 0.05 g of extract in 0.5 ml of solution. Extract solutions were also made containing approximately 0.4 g of extract in 0.5 ml of solution. The extract solutions were transferred to separate centrifuge tubes each containing 0.5 g of Inactivated Demineralized Bone Matrix (IDBM) inactivated by soaking in 4M Guanidine HCl Solution for 48-72 hours and then rinsing with water (complete transfer may require serial rinsing of the extract tubes). The extract/IDBM solutions were then mixed thoroughly and the IDBM was allowed to soak in the extract for 10-20 minutes. Each tube was labeled, frozen at −80 degree freezer, and lyophilized.

The extract loaded IDBM was weighed out into 15-20 mg aliquots for implantation (a minimum of 8 implants).

### Example 2:

### Surgical Implantation of Growth Factors

Young Sprague-Dawley rats (200-410 g) were anesthetized with 86 mg/kg Ketamine, and 13 mg/kg Xylazine administered intramuscularly (in the thigh). A parallel-mid-line incision was made from the tip of the sternum to just above the groin. The lateral aspects of the rectus abdominus were accessed by blunt dissection to either side of the animal. Three short incisions were made in the muscle on each side and the implants inserted, followed by 1 to 2 stitches with Prolene 3-0 suture (to mark the location and prevent the ejection of the implant mass). One negative control (IDBM without extract) as well as two experimental compositions were inserted on each side. Implant locations were random except that each rat had negative control on each side.

Animals were returned to their cages and provided food and water ad-lib. All members of the study group were kept for 4 weeks. After 4 weeks, animals were sacrificed by asphyxiation with Nitrogen. The rectus abdominus was removed by sharp dissection, removing as much tissue as possible.

### Example 3:

### Histological Analysis of Explants

Each muscle obtained from the procedure outlined in Example 2 above was notched to mark the superior side of the animal and placed into a labeled petri dish. Two of each variety of explant were removed from the muscle and fixed in 10% buffered formalin. Histological sections were taken and consecutive sections were stained with H&E and Masson's trichrome stain. These histological samples were examined by a qualified technician.

The samples were given a score from 0-4 based on the new formation of bone and/or cartilage: 0 represents no new formation in the implant area, 1 represents up to 25% new formation, 2 represents up to 50% new formation, 3 represents up to 50%, and 4 represents 100%. The results of the histological analysis is outlined in the following table.

| Group | Histo Score | Minimum Score | Maximum Score |
|---|---|---|---|
| 0.8 g/cc marrow | 0 ± 0 | 0 | 0 |
| 0.1 g/cc marrow | 0.4 ± 0.5 | 0 | 1 |
| 0.4 g/cc muscle | 0.7 ± 0.5 | 0 | 1 |
| 0.05 g/cc muscle | 1.7 ± 1.0 | 0 | 3 |
| IDBM (-control) | 0 ± 0 | 0 | 0 |

### Example 4:

### Extraction of Growth Factors from Platelets

Obtained outdated apheretically purified platelets (platelets present in 60-70 ml plasma). Keep platelets at 4° C. Combined donor platelets into 500 ml centrifuge tubes. Centrifuged tubes at 8000× g 20 minutes at 4° C. Removed plasma. Added 20 volumes of ice cold sterile saline to platelets and gently resuspended pellet. This step is to remove as much plasma/serum components as possible. Re-centrifuged at 8000 g 20 min at 4° C. to repellet platelets. To platelet pellet, added 10 volumes extraction buffer and agitated overnight at 4° C. (12-16 hours). Pelleted lysed platelet material by centrifugation at 12,000 rpm 20 minutes 4° C. Removed platelet extract.

The inventors found that washing the platelets did not remove any of the growth factor activity from the platelets. If extract is prepared using high salt buffer, it only needs to be sterile filtered and diluted 10 fold to use. If acid ethanol is used, ethanol has to be removed by lyophilization.

### Acid Ethanol

45% Ethanol containing 150 μl concentrated HCl for every 50 ml of solution

### High salt buffer

100 mM NaH₂PO₄

1.5M NaCl

pH 7.4

For related materials and methods (as well as terms and techniques) commonly used in the art, please see, for example, WO 98/40113, U.S. Pat. No. 4,294,753, U.S. Pat. No. 5,422,340. The disclosure of all patents and publications cited in this application are incorporated by reference in their entirety to the extent that their teachings are not inconsistent with the teachings herein.

What is claimed is:

1. A method of obtaining growth factors from tissue comprising the steps of:
(a) obtaining tissue; and
(b) extracting one or more growth factors from said tissue.

2. The method of claim 1 wherein said growth factors are osteogenic.

3. The method of claim 1 wherein said tissue is selected from the group consisting of bone, bone marrow, neural tissue, fibrous connective tissue, cartilage, muscle, vasculature, skin, adipose tissue, and glandular tissue.

4. The method of claim 1 wherein said tissue is muscle or bone marrow.

5. The method of claim 1 wherein said tissue is skin.

6. The method of claim 1 wherein said extracting step comprises treating said tissue with a solubilizing agent, and sequestering said one or more growth factors.

7. The method of claim 6 wherein said solubilizing agent is Guanidine HydroChloride, Urea, Triton X, Sodium Dodecyl Sulfate, or combinations thereof.

8. One or more growth factors obtained by a process according to claim 1.

9. A method of treating a defect or injury in a patient comprising implanting into said patient the growth factor of claim 8.

10. The method of claim 9 wherein said growth factor is combined with a suitable carrier.

11. The method of claim 10 wherein said suitable carrier is gelatin, glycerol, collagen, amylopectin, agarose, dextran, inulin, hyaluronic acid, cellulose, albumin, cellulose and derivatives thereof, polyhydroxy compounds, biodegradable polymers, polylactic acid, polyglycolic acid, polyvinyl compounds, polycoprolactone, degradable polyesters, polysulfones, polycarbonates, polyolefins, polyphosphasines polyacrylates, polyamides, polycyanoacrylates, or combinations therof.

12. The method of claim 10 wherein said carrier is an allograft or xenograft.

13. A growth factor composition comprising a growth factor derived from tissue and a carrier.

14. An osteogenic growth factor composition comprising an osteogenic growth factor obtained from nonbone tissue; a carrier component; and one or more other osteogenic components.

15. The osteogenic growth factor composition of claim 14 wherein said one or more other osteogenic components comprise growth factors obtained from bone; carrier associated mineralized particles; morsellized skin or other tissue; Fibrin powder; Fibrin/plasminogen glue; bioactive glass; bioactive ceramic; Demineralized Bone Matrix (DBM)/glycerol; cortico cancellous chips (CCC); DBM/pleuronic F127; DBM/CCC/F127; human tissue associated with polyesters polyhydroxy compounds, polyvinyl compounds, polyamino compounds, or polycarbonate compounds; and combinations thereof.

16. An osteogenic growth factor extracted from muscle.

17. Platelet rich plasma obtained from an allogenic or xenogenic cadaveric donor tissue source.

18. The platelet rich plasma of claim 17, wherein the platelet rich plasma is obtained from blood that has been removed from living or cadaveric donors.

19. A method of obtaining platelet rich plasma comprising the steps of:
(a) procuring blood that has been removed from living or cadaveric donors, or both; and
(b) separating platelet rich plasma from other blood components.

20. The method of claim 19, wherein said separating comprises centrifuging said blood.

21. A growth factor composition comprising one or more growth factors that have been extracted from allogenic or xenogenic platelet rich plasma.

22. The growth factor composition of claim 21 comprising PDGF, PAGF, PEGF, TGF-beta, or combinations thereof.

23. The growth factor composition of claim 21, wherein said platelet rich plasma is obtained from blood that has been removed from living or cadaveric donors.

23. An article of manufacture comprising a container and a growth factor composition disposed within said container.

24. The article of manufacture of claim 23, wherein said container is a sealed bottle, vial, syringe or bag.

25. A method of repairing a wound, defect or other injury comprising contacting an implant with PRP obtained from allogenic or xenogenic sources, or both; and implanting said implant in a patient in need thereof.

26. A method of repairing a wound, defect or other injury comprising contacting an implant with one or more growth factors extracted from PRP obtained from allogenic or xenogenic sources, or both; and implanting said implant in a patient in need thereof.

27. A method of treating a defect or injury in a patient comprising infusing an implant with the one or more growth factors of claim 8, and implanting said implant into said patient.

28. The method of claim 27 wherein said one or more growth factors are derived from cadaveric tissue.

29. The method of claim 27 wherein said implant is comprised of bone (cortical and/or cancellous), mineralized collagen, Bio Oss, Norian SRS, collagraft, osteoset, hydroxyapatite, bioglass, aluminates, tricalciumphosplate, calcium sulphate and calcium phosplate, polymeric materials such as acrylic ester polymers and lactic acid polymers, or glycosaminoglycan (GAG), or combinations thereof.

30. The method of claim 29 wherein said implant is comprised of a mono-, di-, or tri-calcium phosphate composition, or combinations thereof.

31. A biomedical implant infused with one or more growth factors derived from cadaveric, nonbone tissue.

32. The biomedical implant of claim 31 wherein said implant is comprised of bone (cortical and/or cancellous), mineralized collagen, Bio Oss, Norian SRS, collagraft, osteoset, hydroxyapatite, bioglass, aluminates, tricalciumphosplate, calcium sulphate and calcium phosplate, polymeric materials such as acrylic ester polymers and lactic acid polymers, or glycosaminoglycan (GAG), or combinations thereof.

33. The biomedical implant of claim 32 wherein said implant is comprised of a mono-, di-, or tri-calcium phosphate composition, or combinations thereof.

34. A biomedical implant comprised of a calcium phosphate composition, wherein said implant is infused with one or more growth factors derived from cadaveric tissue.

35. A growth factor composition comprising one or more growth factors derived from cadaveric tissue and a carrier;
wherein said carrier comprises growth factors obtained from bone; carrier associated mineralized particles; morsellized skin or other tissue; Fibrin powder; Fibrin/plasminogen glue; bioactive glass; bioactive ceramic; Demineralized Bone Matrix (DBM)/glycerol; cortico cancellous chips (CCC); DBM/pleuronic F127; DBM/CCC/F127; human tissue associated with polyesters polyhydroxy compounds, polyvinyl compounds, polyamino compounds, or polycarbonate compounds; and combinations thereof.

36. A method of extracting growth factors from platelets comprising the steps of:
obtaining a sample of platelets apheretically separated from donor blood;
centrifuge platelets to separate platelets from plasma; and
agitate platelets in an extraction buffer to lyse platelets.

37. The method of claim 36 further comprising centrifuging agitated platelets.

38. The method of claim 36, wherein said extraction buffer is acid ethanol or high salt buffer.
